(19) United States
(12) Patent Application Publication
(10) Pub. No.: US 2009/0215763 A1
(43) Pub. Date: Aug. 27, 2009

(54) SUBSTITUTED BENZOXAZINONES

(75) Inventors: Daniel Holsworth, San Diego, CA (US); William Park, Westerly, RI (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: P&H Therapeutics, Inc., Providence, RI (US)

(21) Appl. No.: 12/392,992

(22) Filed: Feb. 25, 2009

Related U.S. Application Data
(60) Provisional application No. 61/031,269, filed on Feb. 25, 2008.

(51) Int. Cl. A61K 31/5365; C07D 498/04; A61P 9/12

(52) U.S. Cl. 514/230.5; 544/105

(57) ABSTRACT

The present invention provides substituted oxazinone compounds, such as substituted benzoxazinones, which exhibit potent renin inhibition activities.

### CROSS-REFERENCES TO RELATED APPLICATIONS

This application claims priority to U.S. Provisional Patent Application No. 61/031,269 filed Feb. 25, 2008, which application is incorporated herein by reference in its entirety and for all purposes.

### BACKGROUND OF THE INVENTION

Hypertension is a leading risk factor for cardiovascular disease, such as congestive heart failure, stroke, and myocardial infarction. Renin is an endopeptidase (molecular weight about 40,000) produced and secreted by the juxtaglomerular cells of the kidney, which cleaves the naturally-occurring plasma glycoprotein and antiotensinogen. Renin cleaves angiotensinogen, its protein substrate, to split off the hemodynamically-inactive N-terminal decapeptide, angiotensin I, which is converted in the lungs, kidney or other tissue by angiotensin-converting enzyme to the potent pressor octapeptide, angiotensin II. Angiotensin II is known to be a potent pressor substance, i.e., a substance that is capable of inducing a significant increase in blood pressure, and is believed to act by causing the constriction of blood vessels and the release of the sodium-retaining hormone aldosterone from the adrenal gland. Thus, the renin-angiotensinogen system has been implicated as a causative factor in certain forms of hypertension and congestive heart failure (Stanton, Journal of the Renin-Angiotensin-Aldosterone System 2003, 4, 6; and Rosenberg, et al. Antihypertensive Drugs 1997, 77).

Inhibitors of angiotensin I converting enzyme have proven useful in the modulation of the renin-angiotensin system. Consequently, specific inhibitors of the limiting enzymatic step that ultimately regulates angiotensin II production, the action of renin on its substrate, are sought as effective therapeutic agents in the treatment of hypertension, and congestive heart failure. However, attempts to inhibit renin have been centered on using high molecular weight transition state mimetics based on the angiotensinogen backbone. These peptidomimetic inhibitors suffered from poor PK properties such as low oral bioavailability, short duration of action, and/or cost of synthesis.

Therefore, there is a need to develop small molecule renin inhibitors that have an improved potency and an optimized renin inhibitory activity. The present invention meets these and other needs.

### BRIEF SUMMARY OF THE INVENTION

In one aspect, the present invention provides a compound having formula (I):

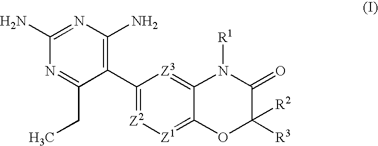

wherein
Z¹, Z² and Z³ are each independently selected from the group consisting of N, CH, C—CH₃ and C—OCH₃;
R¹ is a member selected from the group consisting of —(CH₂)ₙ—OR^{a}, —(CH₂)ₙ—CF₃, —(CH₂)ₙ—CN, —(CH₂)ₙ—NHR^{b} and —(CH₂)ₙ—C(O)NHR^{a}, wherein R^{a} is a member selected from the group consisting of H, C₁₋₃ alkyl, C₁₋₃ hydroxyalkyl and C₁₋₃ haloalkyl; R^{b} is a member selected from the group consisting of —C(O)R^{c}, —C(O)OR^{c} and —S(O)₂R^{c}; and subscript n is an integer from 1-3, wherein R^{c} is selected from the group consisting of C₁₋₆ alkyl, C₁₋₆ hydroxyalkyl and C₁₋₆ haloalkyl; and wherein
(i) when at least one of Z¹, Z² and Z³ is N;
   R² and R³ are each independently selected from the group consisting of C₁₋₆ alkyl, C₁₋₆ haloalkyl, substituted or unsubstituted pyridyl, substituted and unsubstituted phenyl, and substituted or unsubstituted piperidinyl;
(ii) when each of Z¹, Z² and Z³ is CH or C—CH₃;
   R² is a member selected from the group consisting of methyl and trifluoromethyl; and
   R³ is a member selected from the group consisting of C₃₋₆ branched alkyl, C₃₋₆ branched haloalkyl, substituted or unsubstituted pyridyl, substituted and unsubstituted phenyl, and substituted or unsubstituted piperidinyl; wherein when R³ is substituted and unsubstituted phenyl, then R² is trifluoromethyl; or
(iii) when Z¹ is C—OCH₃;
   R² and R³ are each independently selected from the group consisting of C₁₋₆ alkyl, C₁₋₆ haloalkyl, substituted or unsubstituted pyridyl, substituted and unsubstituted phenyl, and substituted or unsubstituted piperidinyl;
wherein the substituents on said pyridyl, phenyl and piperidinyl groups are selected from the group consisting of halo, C₁₋₆ alkoxy, C₁₋₆ alkyl, C₁₋₆ haloalkyl, C₁₋₆ alkylamino and di C₁₋₆ alkylamino;
   and pharmaceutically acceptable salts or solvates thereof.

In another aspect, the present invention provides a pharmaceutical composition. The composition includes a pharmaceutically acceptable excipient and a compound of formula (I).

In yet another aspect, the present invention provides a method of treating renin-mediated diseases or conditions. The method includes administering to a subject in need of such treatment an effective amount of a compound of formula (I).

### BRIEF DESCRIPTION OF THE DRAWINGS

FIG. 1 illustrates one synthetic approach to certain substituted benzoxazinones according to an embodiment the present invention.

FIG. 2 illustrates another synthetic approach to certain other substituted benzoxazinones according to an embodiment the present invention.

### DETAILED DESCRIPTION OF THE INVENTION

### I. Definitions

The term “alkyl”, by itself or as part of another substituent, means, unless otherwise stated, a straight or branched chain saturated hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. For each of the definitions herein (e.g., alkyl, alkoxy, alkylamino, haloalkyl), when a prefix is not included to indicate the number of main chain carbon atoms in an alkyl portion, the radical or portion thereof will have 12 or fewer main chain carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached having from 0-2 additional heteroatoms selected from N, O or S. Accordingly, a group represented as —NR′R″ is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom.

The term “haloalkyl,” is meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, and the like.

The term “hydroxyalkyl” is meant to include at least one hydroxyl group appended to the parent molecular moiety through an alkyl group, as defined herein. Representative examples of hydroxyalkyl include, but are not limited to, hydroxymethyl, 2-hydroxyethyl, 2-methyl-2-hydroxyethyl, 3-hydroxypropyl, 2,3-dihydroxypentyl, and 2-ethyl-4-hydroxyheptyl.

As used herein, the term “heteroatom” is meant to include oxygen (O), nitrogen (N) and sulfur (S).

Certain compounds of the present invention can exist in unsolvated forms as well as solvated forms, including hydrated forms. “Hydrate” refers to a complex formed by combination of water molecules with molecules or ions of the solute. “Solvate” refers to a complex formed by combination of solvent molecules with molecules or ions of the solute. The solvent can be an organic compound, an inorganic compound, or a mixture of both. Solvate is meant to include hydrate. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Certain compounds of the present invention possess asymmetric carbon atoms (optical centers); the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and deleterious to the recipient thereof.

The term “pharmaceutically acceptable excipient or carrier” means one or more excipients that are useful in preparing a pharmaceutical composition. Excipients are generally safe, non-toxic and neither biologically nor otherwise undesirable, and include excipients that are acceptable for veterinary use as well as human pharmaceutical use. “A pharmaceutically acceptable carrier” as used in the specification and claims includes both one and more than one such carrier.

The term “therapeutically effective amount” refers to the amount of a compound that, when administered to a mammal for preventing or treating a disease, is sufficient to effect such prevention or treatment for the disease. The “therapeutically effective amount” will vary depending on the compound, the disease and its severity and the age, weight, etc., of the mammal to be treated.

As used herein, “administering” means oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “subject” means mammals, including, without limitation, humans, domestic animals (e.g., dogs or cats), farm animals (cows, horses, or pigs), goats, monkeys, rabbits, mice, and laboratory animals.

### II. General

The present invention relates to substituted oxazinones that are aspartic acid protease inhibitors in general and renin inhibitors in particular. Advantageously, the compounds have improved potencies and optimized renin inhibitory activities.

### III. Compounds

In one aspect, the present invention provides a compound having formula (I):

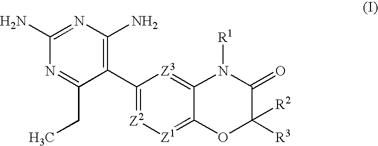

and pharmaceutically acceptable salts or solvates thereof. The variables Z¹, Z², Z³, R¹, R² and R³ are as defined in the Summary of Invention. In one embodiment, the stereochemical configuration at the carbon atom bearing R² and R³ is racemic. In another embodiment, the stereochemical configuration at the carbon atom bearing R² and R³ is (R). Preferably, the stereochemical configuration at the carbon atom bearing R² and R³ is (S).

In a group of embodiments of compounds of formula (I), R¹ is —(CH₂)ₙ—NHC(O)OR^{c}, —(CH₂)ₙ—NHC(O)NHR^{c}, —(CH₂)ₙ—NHC(O)N(R^{c})₂, or —(CH₂)ₙ—OR^{c}, wherein the subscript n is 1, 2 or 3. In certain instances, R¹ is —CH₂CH₂NHC(O)OR^{c}, —CH₂CH₂NHC(O)NHR^{c}, —CH₂CH₂NHC(O)N(R^{c})₂ or —CH₂CH₂CH₂OR^{c}. In other instances, R¹ is —(CH₂)ₙ—NHC(O)OC₁₋₆alkyl, —(CH₂)ₙ—NHC(O)OC₁₋₆haloalkyl or —(CH₂)ₙ—OC₁₋₆haloalkyl. Preferably, R¹ is —CH₂CH₂NHC(O)OC₁₋₆alkyl. In some preferred embodiments, R¹ is —(CH₂)₂NHC(O)OCH₃, —(CH₂)₂NHC(O)OCF₃, —(CH₂)₂NHC(O)NHCH₃, —(CH₂)₂NHC(O)N(CH₃)₂ or —(CH₂)₃OCF₃. Z¹, Z² and Z³ are as defined in formula (I).

In a preferred group of embodiments of compounds having formula (I) as set forth in proviso (i) of the summary of invention, Z² and Z³ are each independently CH, C—CH₃ or C—OCH₃ and Z¹ is N. More preferably, Z¹ is N, Z² and Z³ are CH, or Z¹ is N, Z² and Z³ are CH or C—CH₃, or Z¹ is N, Z² and Z³ are each independently CH, C—CH₃ or C—OCH₃. In certain instances, Z¹ is N, Z² is CH and Z³ is C—CH₃ or C—OCH₃. In other instances, Z¹ is N, Z² is C—CH₃ or C—OCH₃ and Z³ is CH. In still other instances, Z¹ is N, Z² is N and Z³ is CH, C—CH₃ or C—OCH₃. Other variables are as defined in formula (I) or any of the embodiments above.

In one group of embodiments of compounds having formula (I) as set forth in proviso (i) of the summary of invention, Z¹ and Z² are each independently CH, C—CH₃ or C—OCH₃ and Z³ is N. In certain instances, Z³ is N, Z¹ and Z² are CH, or Z³ is N, Z¹ and Z² are C—CH₃, or Z³ is N, Z¹ and Z² are C—OCH₃. In yet other instances, Z³ is N, Z¹ is CH and Z² is C—CH₃ or C—OCH₃. In still other instances, Z³ is N, Z¹ is C—CH₃ or C—OCH₃ and Z² is CH. In other instances, Z¹ is CH, C—CH₃ or C—OCH₃, Z² is N and Z³ is N. Other variables are as defined in formula (I) or any of the embodiments above.

In another group of embodiments as set forth in proviso (i) of the summary of invention, Z¹ and Z³ are each independently N and Z² is CH, C—CH₃ or C—OCH₃. In certain instances, Z¹ and Z³ are N and Z² is CH, or Z¹ and Z³ are N and Z² is C—CH₃, or Z¹ and Z³ are N and Z² is C—OCH₃. Other variables are as defined in formula (I) or any of the embodiments above.

In yet another group of embodiments as set forth in proviso (i) of the summary of invention, Z¹ and Z³ are each independently CH, C—CH₃ or C—OCH₃ and Z² is N. In certain instances, Z¹ and Z³ are CH, C—CH₃ or C—OCH₃ and Z² is N. In certain other instances, Z² is N, Z¹ and Z³ are CH. In yet certain other instances, Z² is N, Z¹ is CH and Z³ is C—CH₃ or C—OCH₃. In still certain other instances, Z² is N, Z¹ is C—CH₃ or C—OCH₃ and Z³ is CH. Other variables are as defined in formula (I) or any of the embodiments above.

In still another group of embodiments as set forth in proviso (i) of the summary of invention, Z¹, Z² and Z³ are N. Other variables are as defined in formula (I) or any of the embodiments above.

In one embodiment, Z¹ is N. In some preferred embodiments within proviso (i), Z¹ is N and R¹ is —(CH₂)ₙNHC(O)OR^{c}, —(CH₂)ₙNHC(O)NHR^{c}, —(CH₂)ₙNHC(O)N(R^{c})₂ or —(CH₂)ₙOR^{c}. In other embodiments, Z¹ is N, R² is C₁₋₆alkyl and R³ is substituted or unsubstituted phenyl. In a preferred embodiment, Z¹ is N, Z² and Z³ are CH. All the other substitutents are as defined in formula (I) or any of the embodiments as defined by proviso (i) above.

In a group of preferred embodiments within proviso (i), Z¹ is N, R² is selected from the group consisting of methyl and trifluoromethyl and R³ is substituted or unsubstituted pyridyl. In certain instances, R³ is selected from the group consisting of substituted or unsubstituted 3-pyridyl and substituted or unsubstituted 4-pyridyl. In one instance, R³ is selected from the group consisting of unsubstituted 3-pyridyl and unsubstituted 4-pyridyl. At each occurrence, the variables Z², Z³ and R¹ are as defined in formula (I) or any of the embodiments above.

In another group of preferred embodiments within proviso (i), Z¹ is N, R² is methyl or —CF₃ and R³ is substituted or unsubstituted phenyl. In certain instances, R³ is di-substituted phenyl. In other instances, R³ is dihalophenyl or di(C₁₋₆haloalkyl)phenyl. In yet other instances, R³ is 3,5-difluorophenyl, 2,3-difluorophenyl, 2,4-difluorophenyl, 2,5-difluorophenyl, 2,6-difluorophenyl, 3,4-difluorophenyl, 3,6-difluorophenyl or 4,5-difluorophenyl. In each occurrence, the variables Z², Z³ and R¹ are as defined in formula (I) or any of the embodiments above.

In one group of embodiments of compounds having formula (I) as set forth in proviso (ii) of the summary of invention, Z¹, Z² and Z³ are each independently CH or C—CH₃. In a preferred embodiment, Z¹, Z² and Z³ are CH. In certain instances, Z¹, Z² and Z³ are C—CH₃. In other instances, Z¹ is CH, Z² is C—CH₃ and Z³ is CH or C—CH₃. In yet other instances, Z¹ is CH, Z² is CH or C—CH₃ and Z³ is C—CH₃. In still other instances, Z¹ is C—CH₃, Z² is CH and Z³ is C—CH₃. In other instances, Z¹, Z² and Z³ are C—CH₃. Other variables are as defined in formula (I) above.

In one embodiment, Z¹ is CH. In another embodiment, Z¹ is C—CH₃. Z² and Z³ are as defined in formula (I).

In one group of embodiments, Z¹ is CH, R² is selected from the group consisting of methyl and trifluoromethyl and R³ is substituted or unsubstituted pyridyl. In certain instances, R³ is selected from the group consisting of substituted or unsubstituted 3-pyridyl and substituted or unsubstituted 4-pyridyl. In other instances, R³ is selected from the group consisting of unsubstituted 3-pyridyl and unsubstituted 4-pyridyl. At each occurrence, the variables Z² and Z³ are as defined in formula (I) or any of the embodiments within proviso (ii) above. R¹ is as defined in formula (I) or any of the embodiments above.

In another group of embodiments, Z¹ is CH, R² is selected from the group consisting of methyl and trifluoromethyl, and R³ is substituted or unsubstituted pyridyl. In certain instances, R³ is selected from the group consisting of substituted or unsubstituted 3-pyridyl and substituted or unsubstituted 4-pyridyl. In other instances, R³ is selected from the group consisting of unsubstituted 3-pyridyl and unsubstituted 4-pyridyl. At each occurrence, the variables Z² and Z³ are as defined in formula (I) or any of the embodiments within proviso (ii) above. R¹ is as defined in formula (I) or any of the embodiments above.

In one group of embodiments, Z¹ is CH, R² is trifluoromethyl, and R³ is substituted or unsubstituted phenyl. In certain instances, the substituted phenyl is selected from the group consisting of 3-methoxyphenyl and 4-methoxyphenyl. At each occurrence, the variables Z² and Z³ are as defined in formula (I) or any of the embodiments as defined by proviso (ii) above. R¹ is as defined in formula (I) or any of the embodiments above.

In one group of embodiments, Z¹ is CH, R² is selected from the group consisting of methyl and trifluoromethyl, and R³ is selected from the group consisting of C₃₋₆ branched alkyl and C₃₋₆ branched haloalkyl. In certain instances, R³ is isopropyl or tert-butyl. At each occurrence, the variables Z² and Z³ are as defined in formula (I) or any of the embodiments as defined by proviso (ii) above. R¹ is as defined in formula (I) or any of the embodiments above.

In one group of embodiments, Z¹ is CH, R² is selected from the group consisting of methyl and trifluoromethyl; and R³ is substituted or unsubstituted piperidinyl. In certain instances, R³ is substituted or unsubstituted 4-piperidinyl. In one occurrence, R³ is N-methylpiperidin-4-yl. At each occurrence, the variables Z² and Z³ are as defined in formula (I) or any of the embodiments as defined by proviso (ii) above. R¹ is as defined in formula (I) or any of the embodiments above.

In one group of embodiments of compounds of formula (I) as set forth in proviso (iii) of the summary of the invention, Z¹ is C—OCH₃, R² is selected from the group consisting of C₁₋₆ alkyl and C₁₋₆ haloalkyl; and R³ is selected from the group consisting of substituted or unsubstituted pyridyl, substituted and unsubstituted phenyl, and substituted or unsubstituted piperidinyl. In certain instances, R² is selected from the group consisting of methyl and trifluoromethyl; and R³ is substituted or unsubstituted pyridyl. At each occurrence, the variables Z² and Z³ are as defined in formula (I) or any of the embodiments as defined by proviso (ii) above. R¹ is as defined in formula (I) or any of the embodiments above.

In a group of embodiments, the compounds of formula (I) have subformula (Ia):

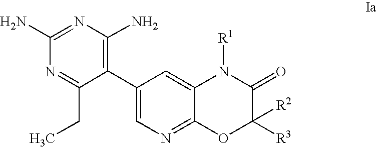

wherein the substituents R¹, R² and R³ are as defined in formula (I) or any of the embodiments within proviso (i) above. The stereochemical configuration of the carbon atom bearing R² and R³ is (S), (R) or racemic, preferably (S).

In another group of embodiments, the compounds of formula (I) have subformula (Ib):

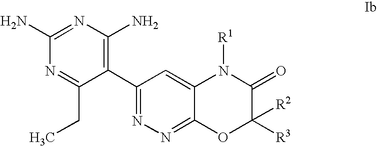

wherein the substituents R¹, R² and R³ are as defined in formula (I) or any of the embodiments as defined by proviso (i) above. The stereochemical configuration of the carbon atom bearing R² and R³ is (S), (R) or racemic, preferably (S).

In yet another group of embodiments, the compounds of formula (I) have subformula (Ic):

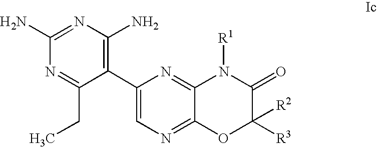

wherein the substituents R¹, R² and R³ are as defined in formula (I) or any of the embodiments as defined by proviso (i) above. The stereochemical configuration of the carbon atom bearing R² and R³ is (S), (R) or racemic, preferably (S).

In still another group of embodiments, the compounds of formula (I) have subformula (Id):

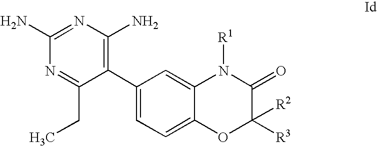

wherein the substituents R¹, R² and R³ are as defined in formula (I) or any of the embodiments as defined by proviso (ii) above. The stereochemical configuration of the carbon atom bearing R² and R³ is (S), (R) or racemic, preferably (S).

In one embodiment, the present invention provides fumarate salts of the compounds of formulas (I), Ia, Ib, Ic or Id. In another embodiment, the present invention provides hydrochloric acid salts of the compounds of formula (I), Ia, Ib, Ic or Id.

The invention provides a compound of formula (I):

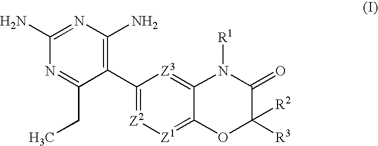

or a pharmaceutically acceptable salt or solvate thereof;
Z¹, Z² and Z³ are each independently selected from the group consisting of N, CH, C—CH₃ and C—OCH₃; R¹ is a member selected from the group consisting of —(CH₂)ₙ—OR^{a}, —(CH₂)ₙ—CF₃, —(CH₂)ₙ—CN, —(CH₂)ₙ—NHR^{b} and —(CH₂)ₙ—C(O)NHR^{a}, wherein R^{a} is a member selected from the group consisting of H, C₁₋₃ alkyl, C₁₋₃ hydroxyalkyl and C₁₋₃ haloalkyl; R^{b} is a member selected from the group consisting of —C(O)R^{c}, —C(O)OR^{c} and —S(O)₂R^{c}; and subscript n is an integer from 1-3, wherein R^{c} is selected from the group consisting of C₁₋₆ alkyl, C₁₋₆ hydroxyalkyl and C₁₋₆ haloalkyl; and wherein
(i) when at least one of Z¹, Z² and Z³ is N;

R² and R³ are each independently selected from the group consisting of C₁₋₆ alkyl, C₁₋₆ haloalkyl, substituted or unsubstituted pyridyl, substituted and unsubstituted phenyl, and substituted or unsubstituted piperidinyl;

(ii) when each of Z¹, Z² and Z³ is CH or C—CH₃;

R² is a member selected from the group consisting of methyl and trifluoromethyl; and

R³ is a member selected from the group consisting of C₃₋₆ branched alkyl, C₃₋₆ branched haloalkyl, substituted or unsubstituted pyridyl, substituted and unsubstituted phenyl, and substituted or unsubstituted piperidinyl; wherein when R³ is substituted and unsubstituted phenyl, then R² is trifluoromethyl; or

(iii) when Z¹ is C—OCH₃;

R² and R³ are each independently selected from the group consisting of C₁₋₆ alkyl, C₁₋₆ haloalkyl, substituted or unsubstituted pyridyl, substituted and unsubstituted phenyl, and substituted or unsubstituted piperidinyl; wherein the substituents on said pyridyl, phenyl and piperidinyl groups are selected from the group consisting of halo, C₁₋₆ alkoxy, C₁₋₆ alkyl, C₁₋₆ haloalkyl, C₁₋₆ alkylamino and di C₁₋₆ alkylamino.

In a first set of embodiments, the invention provides compounds of formula (I), wherein Z¹ is N. Other variables are as defined in formula (I) above.

In a second set of embodiments, the invention provides a compound of formula (I) or the second set, wherein R¹ is —(CH₂)ₙNHC(O)OR^{c} or —(CH₂)ₙNHC(O)NHR^{c}, —(CH₂)ₙNHC(O)R^{c} or —(CH₂)ₙNHR^{c}. Other variables are as defined in formula (I) above.

In a third set of embodiments, the invention provides a compound of formula (I) or any of sets 1 and 2, wherein R^{c} is —CH₃ or —CF₃. Other variables are as defined in formula (I) above.

In a fourth set of embodiments, the invention provides a compound of formula (I) or any of sets 1, 2 and 3, wherein R¹ is —(CH₂)₂NHC(O)OR^{c} or —(CH₂)₂NHC(O)NHR^{c}, —(CH₂)₂NHC(O)R^{c} or —(CH₂)₃NHR^{c}. Other variables are as defined above.

In a fifth set of embodiments, the invention provides a compound of formula (I) or any of sets 1, 2, 3 and 4, wherein R² is selected from the group consisting of methyl and trifluoromethyl; and R³ is selected from the group consisting of substituted or unsubstituted pyridyl and substituted or unsubstituted phenyl. Other variables are as defined above.

In a sixth set of embodiments, the invention provides a compound of formula (I) or any of sets 1, 2, 3, 4 and 5, wherein R³ is substituted or unsubstituted phenyl. Other variables are as defined above.

In a seventh set of embodiments, the invention provides a compound of formula (I) or any of sets 1, 2, 3, 4, 5 and 6, wherein the substituents on the phenyl group is halo or C₁₋₆haloalkyl. Other variables are as defined above.

In an eighth set of embodiments, the invention provides a compound of formula (I) or any of sets 1, 2, 3, 4, 5, 6 and 7, wherein the substitutents on the phenyl group is fluoro. Other variables are as defined above.

In a ninth set of embodiments, the invention provides a compound of formula (I) or any of sets 1, 2, 3, 4, 5, 6, 7 and 8, wherein R³ is 3,5-difluorophenyl. Other variables are as defined above.

In a tenth set of embodiments, the invention provides a compound of formula (I) or any of sets 1, 2, 3, 4 and 5, wherein R³ is selected from the group consisting of substituted or unsubstituted 3-pyridyl and substituted or unsubstituted 4-pyridyl. Other variables are as defined above.

In an eleventh set of embodiments, the invention provides a compound of formula (I) or any of sets 1, 2, 3, 4 and 5 and 10, wherein R³ is selected from the group consisting of unsubstituted 3-pyridyl and unsubstituted 4-pyridyl.

In a twelfth set of embodiments, the invention provides a compound of formula (I), wherein Z¹ is CH.

In a thirteenth set of embodiments, the invention provides a compound of formula (I) or set 12, wherein Z² and Z³ are CH.

In a fourteenth set of embodiments, the invention provides a compound of formula (I) or set 12 or 13, wherein R² is selected from the group consisting of methyl and trifluoromethyl; and R³ is substituted or unsubstituted pyridyl.

In a fifteenth set of embodiments, the invention provides a compound of formula (I) or any of sets 12, 13 and 14, wherein R³ is selected from the group consisting of substituted or unsubstituted 3-pyridyl and substituted or unsubstituted 4-pyridyl.

In a sixteenth set of embodiments, the invention provides a compound of formula (I) or any of sets 12, 13, 14 and 15, wherein R³ is selected from the group consisting of unsubstituted 3-pyridyl and unsubstituted 4-pyridyl.

In a seventeenth set of embodiments, the invention provides a compound of formula (I) or any of sets 12 or 13, wherein R² is trifluoromethyl; and R³ is substituted or unsubstituted phenyl.

In an eighteenth set of embodiments, the invention provides a compound of any of sets 12, 13 and 17, wherein the substituted phenyl is selected from the group consisting of 3-methoxyphenyl and 4-methoxyphenyl.

In a nineteenth set of embodiments, the invention provides a compound of formula (I) or any of sets 12 or 13, wherein R² is selected from the group consisting of methyl and trifluoromethyl; and R³ is selected from the group consisting of C₃₋₆ branched alkyl and C₃₋₆ branched haloalkyl.

In a twentieth set of embodiments, the invention provides a compound of formula (I) or any of sets 12, 13 and 19, wherein R³ is isopropyl or tert-butyl.

In a 21 st set of embodiments, the invention provides a compound of formula (I) or sets 12 or 13, wherein R² is selected from the group consisting of methyl and trifluoromethyl; and R³ is substituted or unsubstituted piperidinyl.

In a 22nd set of embodiments, the invention provides a compound of formula (I) or any of sets 12, 13 and 21, wherein R³ is substituted or unsubstituted 4-piperidinyl.

In a 23rd set of embodiments, the invention provides a compound of formula (I) or any of sets 12, 13, 21 and 22, wherein R³ is N-methylpiperidin-4-yl.

In a 24th set of embodiments, the invention provides a compound of formula (I), wherein Z¹ is C—OCH₃.

In a 25th set of embodiments, the invention provides a compound of formula (I) or set 24, wherein R² is selected from the group consisting of C₁₋₆ alkyl and C₁₋₆ haloalkyl; and R³ is selected from the group consisting of substituted or unsubstituted pyridyl, substituted and unsubstituted phenyl, and substituted or unsubstituted piperidinyl.

In a 26th set of embodiments, the invention provides a compound of formula (I) or any of sets 24 and 25, wherein R² is selected from the group consisting of methyl and trifluoromethyl; and R³ is substituted or unsubstituted pyridyl.

In a 27th set of embodiments, the invention provides a compound of formula (I) or any of sets 1-26, wherein the stereochemical configuration at the carbon atom bearing R² and R³ is (S).

In a 28th set of embodiments, the invention provides a pharmaceutical composition comprising a pharmaceutically acceptable excipient and a compound of formula (I) or any of sets 1-27.

### Preparation of Compounds

All the compounds described herein including compounds having formulas I, Ia, Ib Ic or Id, compounds of sets 1-28, and compounds as defined in claims 1-29 can be prepared by the methods described in Schemes 1 and 2 hereinbelow. Compounds of the present invention can be prepared using readily available starting materials or known intermediates. Examples of starting materials available from commercial suppliers include, but are not limited to, 4-bromo-2-nitrophenol, 4-bromo-1-fluoro-2-nitrobenzene, 5-bromo-3-nitropyridine-2-ol, 2-hydroxy carboxylic esters, 2-bromo carboxylic esters, 2-substituted racemic 2-hydroxy carboxylic esters, 2-substituted optically active 2-hydroxy carboxylic esters, 2-substituted racemic 2-bromo carboxylic esters and 2-substituted optically active 2-bromo carboxylic esters. Other starting materials can be prepared according to the literature procedures. The 2-hydroxy or 2-bromo carboxylic esters include those where the 2-position is further substituted with one or two non-hydrogen substituents. The 2-hydroxy or 2-bromo carboxylic esters can be either optically active or racemic. Non commercially available 2-hydroxy or 2-bromo carboxylic esters can be synthesized by alkylation or arylation of 2-hydroxy or 2-bromo carboxylic esters, where the hydroxyl groups can be either protected or unprotected. For alkylation, the reaction can be carried out in the presence of a base, such as lithium diisopropylamide (LDA) (see, Williams, et al. J. Org. Chem. 1980, 45, 5082; and Rathke, et al. J. Am. Chem. 1971, 93, 2320). For arylation, the reaction can be carried out in the presence of a copper halide or a palladium complex (see, Lindley, et al. Tetrahedron 1984, 40, 1433-1456; Uno, et al. Synthesis 1985, 506; Hartwig, et al. J. Am. Chem. Soc., 2002, 124, 12557-12565; and Marion, et al. J. Org. Chem., 2006, 71, 3816-3821). The choice of appropriate reaction conditions is within the ability of those of skill in the art.

Optically active 2-hydroxy or 2-bromo carboxylic esters can be readily prepared by using chiral auxiliaries or asymmetric catalysts (see, Hartwig, et al. J. Am. Chem. Soc. 2004, 126, 5182-5191). The choice of appropriate reaction conditions can be readily established by those of skill in the art. For example, (S) or (R)-2-hydroxy carboxylic acid ester can be prepared by nucleophilic addition reaction of dialkylzinc with 2-aryl-oxoacetate in the presence of an (S) or (R) chiral auxiliary, such as (S)—N-benzyl-2-hydroxy-2-phenylacetamide or (R)—N-benzyl-2-hydroxy-2-phenylacetamide.

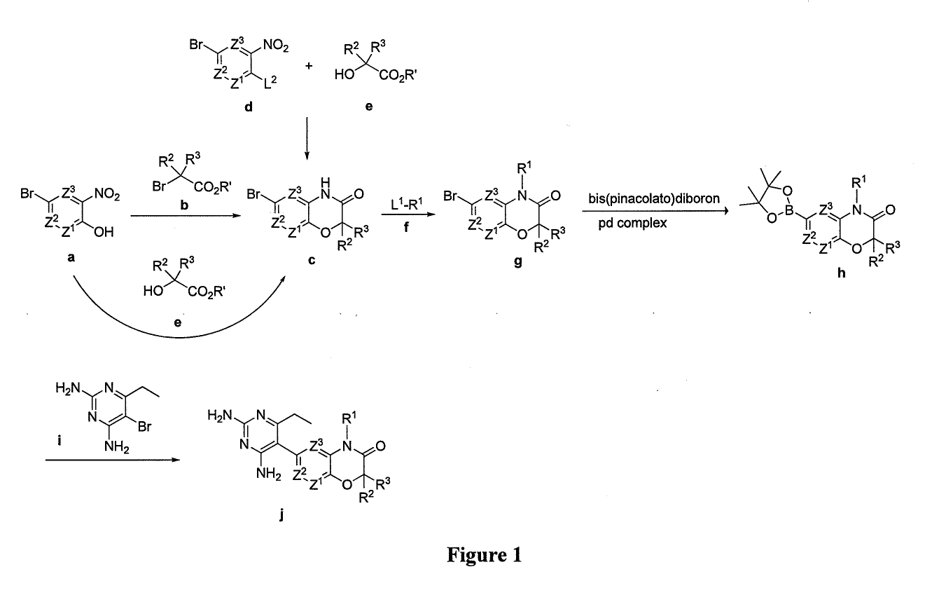

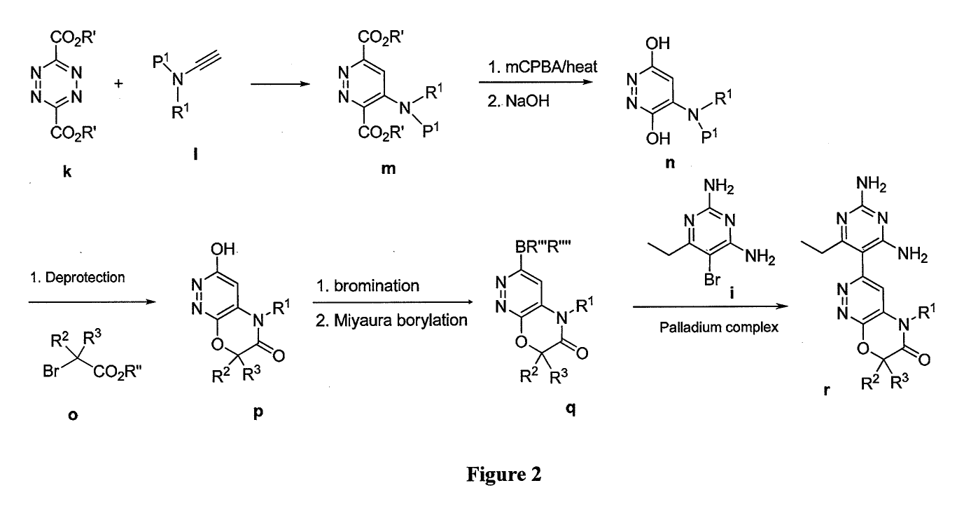

As shown in the examples below, there are a variety of synthetic routes by which a skilled artisan can prepare compounds and intermediates of the present invention. Schemes 1 and 2 (FIGS. 1-2) illustrates two approaches for the synthesis of certain substituted benzoxazinones. Enantiomerically pure substituted benzoxazinones compounds can be obtained using chiral separation techniques known in the art including chiral chromatographies, crystallizations and chiral resolution agents (see, Porter, et al. Pure & Appl. Chem., 1991, 63, 1119-1122; Beesely, et al. Chiral Chromatogrphy, 1st Ed. Wiley, 1999; Harold, J. Am. Chem. Soc. 1955, 77, 2910; and Yoshito, et al. Org. Process Res. Dev.; 2006; 10 (5) pp 905-913). In Schemes 1 and 2, L¹ is a leaving group, L² is a labile group capable of reacting with a nucleophile and R′, R″, R′″ and R″″ are non-interfering substituents. P¹ is an amino protecting group. Examples of protecting groups can be found in T. W. Greene and P. G. Wuts, Protective Groups in Organic Chemistry, (Wiley, 4th ed. 2006), Beaucage and Iyer, Tetrahedron 48:2223-2311 (1992), and Harrison and Harrison et al., Compendium of Synthetic Organic Methods, Vols. 1-8 (John Wiley and Sons. 1971-1996). Representative amino protecting groups include formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC) and the like. R¹, R², R³, Z¹, Z² and Z³ correspond to the respective substituents in formula I.

The reaction schemes 1 and (FIGS. 1 and 2) provides certain synthetic routes that can be followed to access certain substituted benzoxazinones of the present invention. Other routes or modification of the routes presented below would be readily apparent to a skilled artisan and within the scope of the present invention.

Scheme 1 (FIG. 1) shows three synthetic approaches to substituted benzoxazinones. All the approaches produce the common intermediate c. In the first approach, compound a, for example, 4-bromo-2-nitrophenol is reacted with a substituted 2-bromo carboxylic ester b in the presence of a base, such as K₂CO₃, followed by reduction of the nitro group and in-situ cyclization to yield 1,4-benzoxazin-3-ones c. In the second approach, the intermediate compound c can be synthesized by reacting compound d bearing a nitro group and a labile group L² with a substituted 2-hydroxy carboxylic ester e through a nucleophilic aromatic substitution reaction in the presence of a base, such as NaH, followed by reduction of the nitro group and in-situ cyclization. The labile group L² can be a halide such as F. In the third approach, the intermediate compound c can be prepared by reacting compound 1 with a substituted 2-hydroxy carboxylic ester e under a Misunobu coupling condition, followed by reduction of the nitro group and in-situ cyclization. As shown in Scheme 1, substituted oxazinones g can be synthesized by nucleophilic substitution reaction of compound c with compound f in the presence of a base, such as NaH. The bromo group of compound g can be readily converted to the boryl group of compound h by reacting compound g with bis(pinacolato)boron in the presence of a palladium complex under a Miyaura borylation condition. Compound j having formula I can be obtained by Suzuki coupling reaction of 5-bromo-2,4-diamino-6-ethyl-pyrimidine i with compound h, for example, in the presence of a palladium complex. Again, the choice of appropriate reaction conditions can be readily established by those of skill in the art.

Scheme 2 (FIG. 2) shows one synthetic approach to substituted pyridazinooxazinones. The starting material tetrazine dicarboxylate k can be readily prepared from ethyl diazoacetate according to the literature procedures (see, Boger, et al. Org. Synth. 1992, 70, 79; and Boger, et al. J. J. Org. Chem. 1985, 50, 5377). Substituted alkyne l is either commercially available or can be readily prepared according to the literature procedures. The key intermediate m can be prepared by a [4+2] hetero Diels-Alder reaction of a tetrazine dicarboxylate k with an amino protected terminal alkyne 1 (see, Gilchrist, T. L. Heterocyclic Chemistry; Pitman Publishing: London, 1985; Sundberg, R. G. Comprehensive Heterocyclic Chemistry, Vol. IV; Pergamon Press: Oxford, 1984; Boger, D. L. Chem. Rev. 1986, 86, 78; and J. Org. Chem. 1989, 54, 714). Subsequent hydrolysis and hydroxylation of compound m produce amino substituted dihydroxy pyridazine n. The hydroxy substituted pyridazinooxazinone p can be prepared by removing the amino protecting group P¹ of compound n and reacting with a substituted bromo carboxylic ester o. The hydroxy group on the pyridazine ring can be converted to a bromo group under a bromonation condition (see, Wiley, et al. J. Am. Chem. Soc. 1964, 86, 964-65) and the resulting product is further reacted either with bis(pinacolato)boron in the presence of a palladium complex under a Miyaura borylation condition to afford the borylated compound q or alternatively, with a Grignard agent and trimethyl borate to form the borylated compound q (see, Wang, et al. Org. Lett., 2006, 8, 305-307). Compound r having formula I can be obtained by Suzuki coupling reaction of 5-bromo-2,4-diamino-6-ethyl-pyrimidine i with compound q in the presence of a palladium complex, such as a palladium phosphine complex.

### IV. Pharmaceutical Compositions

In accordance with the present invention, a therapeutically effective amount of a compound of any of Formulas (I), (Ia), (Ib), (Ic), (Id) or sets 1-28 or a compound of Examples 8-10 can be used for the preparation of a pharmaceutical composition useful for treating and/or preventing hypertension, congestive heart failure, stroke and myocardial infarction.

The compositions of the invention can include compounds of any of Formulas (I), (Ia), (Ib), (Ic), (Id) or sets 1-28 or a compound of Examples 8-10, pharmaceutically acceptable salts thereof, a hydrate thereof or a hydrolysable precursor thereof. In general, the compound is mixed with suitable carriers or excipient(s) in a therapeutically effective amount. By a “therapeutically effective dose”, “therapeutically effective amount” or, interchangeably, “pharmacologically acceptable dose” or “pharmacologically acceptable amount”, it is meant that a sufficient amount of the compound of the present invention and a pharmaceutically acceptable carrier, will be present in order to achieve a desired result, e.g., alleviating a symptom or complication of hypertension.

The compounds of any of Formulas (I), (Ia), (Ib), (Ic), (Id) or sets 1-28 or a compound of Examples 8-10 that are used in the methods of the present invention can be incorporated into a variety of formulations for therapeutic administration. More particularly, the compounds of any of Formulas (I), (Ia), (Ib), (Ic), (Id) or sets 1-28 or Examples 8-10 can be formulated into pharmaceutical compositions by combination with appropriate, pharmaceutically acceptable carriers or diluents and can be formulated into preparations in solid, semi-solid, liquid or gaseous forms, such as tablets, capsules, pills, powders, granules, dragees, suspensions, gels, slurries, ointments, solutions, suppositories, injections, inhalants and aerosols. As such, administration of the compounds can be achieved in various ways, including oral, buccal, rectal, parenteral, intraperitoneal, intradermal, transdermal, intratracheal administration. Moreover, the compound can be administered in a local manner, in a depot or sustained release formulation. In addition, the compounds can be administered in a liposome.

The compounds of any of Formulas (I), (Ia), (Ib), (Ic), (Id) or sets 1-28 or a compound of Examples 8-10 can be formulated with common excipients, diluents or carriers and compressed into tablets or formulated as elixirs or solutions for convenient oral administration or administered by intramuscular or intravenous routes. The compounds can be administered transdermally and can be formulated as sustained release dosage forms and the like. Compounds of any of Formulas (I), (Ia), (Ib), (Ic), (Id) or sets 1-28 can be administered alone, in combination with each other or they can be used in combination with other known compounds.

Suitable formulations for use in the present invention are found in Remington. The Science and Practice of Pharmacy, 21 st ed., Lippincott Williams & Wilkins, Philadelphia, Pa., 2005, which is incorporated herein by reference. Moreover, for a brief review of methods for drug delivery, see, Langer, Science (1990) 249:1527-1533, which is incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

The pharmaceutical compositions for the administration of the compounds of this invention may conveniently be presented in unit dosage form and may be prepared by any of the methods well known in the art of pharmacy and drug delivery. All methods include the step of bringing the active ingredient into association with the carrier, which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the process or condition of diseases.

The pharmaceutical compositions containing compound of formula I may be in a form suitable for oral use, for example, as tablets, troches, lozenges, aqueous or oily suspensions, dispersible powders or granules, emulsions and self emulsifications as described in U.S. Patent Application 2002-0012680, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Oral solutions can be prepared in combination with, for example, cyclodextrin, PEG and surfactants.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols. Additionally, the compounds can be administered via ocular delivery by means of solutions or ointments. Still further, transdermal delivery of the subject compounds can be accomplished by means of iontophoretic patches and the like. For topical use, creams, ointments, jellies, solutions or suspensions, etc., containing the compounds of the present invention are employed. As used herein, topical application is also meant to include the use of mouth washes and gargles.

Dragee cores are provided with suitable coatings. For this purpose, concentrated sugar solutions can be used, which can optionally contain gum arabic, talc, polyvinyl pyrrolidone, carbopol gel, polyethylene glycol and/or titanium dioxide, lacquer solutions and suitable organic solvents or solvent mixtures. Dyestuffs or pigments can be added to the tablets or dragee coatings for identification or to characterize different combinations of active compound doses.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Alternatively, other deliveries for hydrophobic pharmaceutical compounds can be employed. Liposomes and emulsions are well known examples of delivery vehicles or carriers for hydrophobic drugs. In a presently preferred embodiment, long-circulating, i.e., stealth liposomes can be employed. Such liposomes are generally described in Woodle, et al., U.S. Pat. No. 5,013,556. The compounds of the present invention can also be administered by controlled release means and/or delivery devices such as those described in U.S. Pat. Nos. 3,845,770; 3,916,899; 3,536,809; 3,598,123; and 4,008,719.

The compounds of this invention may also be coupled with a carrier that is a suitable polymer as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the compounds of the invention may be coupled to a carrier that is a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like. In one embodiment of the invention, the compound of the invention is coupled to a polymer or semipermeable polymer matrix that is formed as a stent or stent-graft device.

Certain organic solvents such as dimethylsulfoxide (DMSO) also can be employed, although usually at the cost of greater toxicity. Additionally, the compounds can be delivered using a sustained-release, such as semipermeable matrices of solid hydrophobic polymers containing the therapeutic agent. Various types of sustained-release materials have been established and are well known by those skilled in the art. Sustained-release capsules can, depending on their chemical nature, release the compounds for a few hours up to over 100 days.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin and polymers such as polyethylene glycols.

Pharmaceutical compositions suitable for use in the present invention include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein.

For any compound used in the method of the present invention, a therapeutically effective dose can be estimated initially from cell culture assays or animal models.

Moreover, toxicity and therapeutic efficacy of the compounds described herein can be determined by standard pharmaceutical procedures in cell cultures or experimental animals, e.g., by determining the LD₅₀, (the dose lethal to 50% of the population) and the ED₅₀ (the dose therapeutically effective in 50% of the population). The dose ratio between toxic and therapeutic effect is the therapeutic index and can be expressed as the ratio between LD₅₀ and ED₅₀. Compounds which exhibit high therapeutic indices are preferred. The data obtained from these cell culture assays and animal studies can be used in formulating a dosage range that is not toxic for use in human. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g., Fingl et al. 1975 In: The Pharmacological Basis of Therapeutics, Ch. 1).

### V. Methods of Treating Renin-Mediated Diseases or Conditions

In another aspect, the compounds of the invention can be used for the treatment of renin-mediated diseases or conditions including hypertension and congestive heart failure.

Treatment methods provided herein include, in general, administration to a subject such as a patient an effective amount of one or more compounds provided herein, e.g., orally, nasally or parenterally. Suitable patients include those patients suffering from or susceptible to (i.e., prophylactic treatment) a disorder or disease identified herein. Typical patients for treatment as described herein include mammals, particularly primates, especially humans. Other suitable patients include domesticated companion animals such as a dog, cat, horse, and the like, or a livestock animal such as cattle, pig, sheep and the like.

In general, treatment methods provided herein comprise administering to a subject such as a patient an effective amount of one or more compounds provided herein, for example, compounds of any of formulas I, Ia, Ib, Ic or Id or sets 1-28 or a compound of Examples 8-10. In a preferred embodiment, the compound(s) of the invention are preferably administered to a patient (e.g., a human) orally. The effective amount may be an amount sufficient to modulate the renin-angiotensin system and/or an amount sufficient to reduce or alleviate the symptoms presented by the patient. Preferably, the amount administered is sufficient to yield a plasma concentration of the compound (or its active metabolite, if the compound is a pro-drug) high enough to detectably inhibit renin in vitro. Treatment regimens may vary depending on the compound used and the particular condition to be treated; for treatment of most disorders, a frequency of administration of 4 times daily or less is preferred. In general, a dosage regimen of 2 times daily is more preferred, with once a day dosing particularly preferred. It will be understood, however, that the specific dose level and treatment regimen for any particular patient will depend upon a variety of factors including the activity of the specific compound employed, the age, body weight, general health, sex, diet, time of administration, route of administration, rate of excretion, drug combination (i.e., other drugs being administered to the patient) and the severity of the particular disease undergoing therapy, as well as the judgment of the prescribing medical practitioner. In general, the use of the minimum dose sufficient to provide effective therapy is preferred. Patients may generally be monitored for therapeutic effectiveness using medical or veterinary criteria suitable for the condition being treated or prevented.

The compounds of the present invention can be administered as frequently as necessary, including hourly, daily, weekly or monthly. Frequency of dosage may also vary depending on the compound used and the particular disease treated. However, for treatment of most disorders, a dosage regimen of 4 times daily, three times daily, or less is preferred, with a dosage regimen of once daily or 2 times daily being particularly preferred. The compounds utilized in the pharmaceutical method of the invention are administered at the initial dosage of about 0.0001 mg/kg to about 3000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. In one embodiment, the dosage is about 30 mg/Kg. The dosages, however, may be varied depending upon the requirements of the patient, the severity of the condition being treated, and the compound being employed. For example, dosages can be empirically determined considering the type and stage of disease diagnosed in a particular patient. The dose administered to a patient, in the context of the present invention should be sufficient to effect a beneficial therapeutic response in the patient over time. The size of the dose also will be determined by the existence, nature, and extent of any adverse side-effects that accompany the administration of a particular compound in a particular patient. It will be understood, however, that the specific dose level for any particular patient will depend upon a variety of factors including the activity of the specific compound employed, the age, body weight, general health, sex, diet, time of administration, route of administration, rate of excretion, drug combination (i.e., other drugs being administered to the patient), the severity of the particular disease undergoing therapy, and other factors, including the judgment of the prescribing medical practitioner. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, or via a patch. It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those of skill in the area.

The time-release effect can be obtained by capsule materials that dissolve at different pH values, by capsules that release slowly by osmotic pressure or by any other known means of controlled release.

It can be necessary to use dosages outside these ranges in some cases as will be apparent to those skilled in the art. Further, it is noted that the clinician or treating physician will know how and when to interrupt, adjust or terminate therapy in conjunction with individual patient response.

The pharmaceutical compositions can be administered to the patient in a variety of ways, including topically, parenterally, intravenously, intradermally, intramuscularly, colonically, rectally or intraperitoneally. Preferably, the pharmaceutical compositions are administered parenterally, topically, intravenously, intramuscularly or orally.

In some embodiments, the present invention provides a compound of any of formulas I, Ia, Ib, Ic and Id and sets 1-28 or a compound of Examples 8-10 or a pharmaceutically acceptable salt or solvate thereof, or as defined in any one of claims 1 to 29, for use as a medicament.

In other embodiments, the present invention provides a compound of any of formulas I, Ia, Ib, Ic and Id and sets 1-28 or a compound of Examples 8-10 or a pharmaceutically acceptable salt or solvate thereof, or according to any one of claims 1 to 29, for use in treating renin-mediated diseases or conditions in a mammal. In certain instances, the treatable diseases include, but are not limited to, hypertension and congestive heart failure.

In some embodiments, the present invention provides the use of a compound of any of formulas I, Ia, Ib, Ic and Id and sets 1-28 or a compound of Examples 8-10 or a pharmaceutically acceptable salt thereof, or according to any one of claims 1 to 29, in the manufacture of a medicament for the prevention and/or treatment of renin-mediated diseases or conditions in a mammal. The treatable and/or preventable conditions include hypertension and congestive heart failure.

### VI. Examples

The following examples are offered to illustrate, but not to limit the claimed invention. In the examples below, unless otherwise stated, temperatures are given in degrees Celsius ° C.); operations were carried out at room or ambient temperature (typically a range of from about 18-25° C. or the specified temperature; evaporation of solvent was carried out using a rotary evaporator under reduced pressure (typically, 4.5-30 mmHg) with a bath temperature of up to 60° C.; the course of reactions was typically followed by TLC and reaction times are provided for illustration only; melting points are uncorrected; products exhibited satisfactory ¹H-NMR and/or LC/MS data and yields are provided for illustration only.

### Example 1

### Preparation of (S)-2-acetoxy-2-phenylacetic acid

### 1

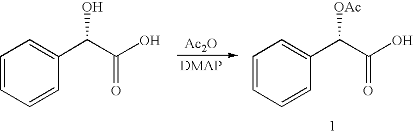

To a round bottom flask charged with the Mandelic acid (15 g, 111 mmol) was added 100 mL of acetic anhydride. After 5 min stirring, 10 mL of anhydrous ether was added and followed by a catalytic amount of DMAP. The resultant reaction mixture was kept at r t for additional 16 h. The reaction mixture was evaporated under reduced pressure to give a amorphous material which in turn diluted with EtOAc (50 mL). The solution was washed with 1N HCl (2×10 mL), water (2×10 mL) and followed by brine solution (10 mL). The org. layer was then dried over MgSO₄ and filtered. The resultant solution was concentrated under reduced pressure. The desired product was isolated by a column chromatography using 20% EtOAc in hexane (15.8 g, 81 mmol). Yield 73%; R_{f} 0.54 (1:1 hex:EtOAc) ¹H NMR (CDCl₃, δ) 11.5 (s, 1H), 7.43 (m, 5H), 5.99 (s, 1H), 2.20 (s, 3H).

Similarly, (R)-2-acetoxy-2-phenylacetic acid can be prepared from (R)-Mandelic acid using the above procedure. Racemic 2-acetoxy-2-phenylacetic acid can be prepared from racemic Mandelic acid using the above procedure.

### Example 2

### Preparation of (S)-(benzylcarbamoyl)(phenyl)methyl acetate

### 2

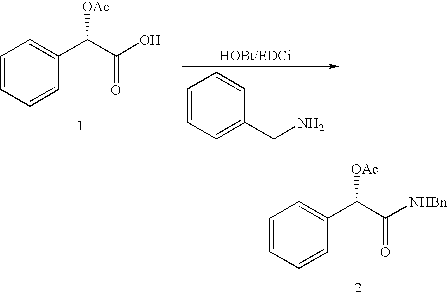

To a DMF solution (7 mL) containing the (S)-2-acetoxy-2-phenylacetic acid (1, 41 mmol) was added EDC and HOBt (2.5 equiv each) and the reaction mixture was stirred for 5 min at ambient temperature prior to the addition of benzyl amine (1.5 equiv). The reaction mixture was then stirred at room temperature for 16 h. The reaction mixture was diluted with EtOAc (25 mL), which was washed with 1N HCl (10 mL×2), sat. NaHCO₃ (10 mL×2), water (10 mL) and followed by brine. The organic layer was then dried over MgSO₄, and filtered. The resultant product solution was concentrated under reduced pressure to give a pale yellow liquid. The crude product was then purified by a column chromatography using a 30% (v/v) EtOAc in hexane as a mobile phase. Yield: 73%; ¹H NMR (CDCl₃, δ) 7.21 (m, 10H), 6.45 (br. s, 1H), 5.08 (s, 1H), 4.43 (q, 2H), 3.64 (s, 1H).

Similarly, (R)-(benzylcarbamoyl)(phenyl)methyl acetate can be prepared from (R)-2-acetoxy-2-phenylacetic acid in accordance with the procedure of compound 2 above. Racemic (benzylcarbamoyl)(phenyl)methyl acetate can be prepared from racemic 2-acetoxy-2-phenylacetic acid using the procedure of compound 2 above.

### Example 3

### Preparation of (S)—N-benzyl-2-hydroxy-2-phenylacetamide

### 3

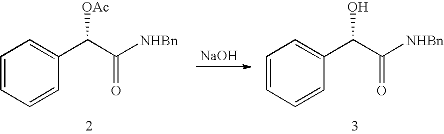

To a MeOH solution (10 mL) containing 0.5N NaOH/MeOH solution (7 mL) was added (S)-(benzyl-carbamoyl)(phenyl)methyl acetate (2, 3.0 g, 10.6 mmol). The reaction was complete less than 1 h at room temperature. The reaction mixture was evaporated under reduced pressure to yield a pale yellow liquid which was re-dissolved in EtOAc (100 mL). The solution was then washed with 1N HCl (15 mL), saturated NaHCO₃ (15 mL) and water. Once washed with brine (5 mL), the organic layer was separated, dried over MgSO₄ and filtered. The filtrate was concentrated under reduced pressure to give a white powder (7.73 mmol). Yield: 73%; ¹H NMR (CDCl₃, δ) 7.23 (m, 10H), 6.45 (br. s, 1H), 5.08 (s, 1H), 4.43 (q, 2H), 3.64 (s, 1H).

The racemic and R-enantiomer of compound 3 can be prepared using similar procedure.

### Example 4

### Preparation of (S)-Ethyl 2-(3,5-difluorophenyl)-2-hydroxypropanoate

### 4

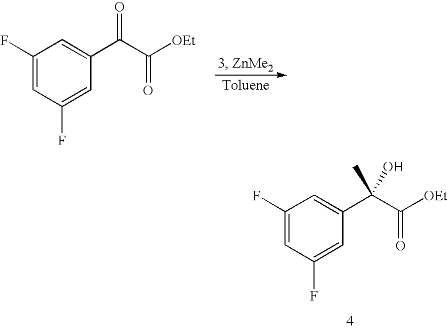

To a toluene solution (2 mL) containing the mandelamide (3, 1.4 g, 5.81 mmol) was added the commercially available substrate, ethyl 2-(3,5-difluorophenyl)-2-oxoacetate and followed by dimethylzinc (8.7 mL of 2M solution in Toluene, 3 equiv, 17.4 mmol) at 0° C. The reaction mixture was stirred at 0° C. for 4 h. After the reaction was quenched with 5 mL of HCl and the same amount of water, EtOAc was added to the mixture. The organic layer was drawn off to a separatory funnel where it was washed with saturated NaHCO₃ (2×10 mL), water (2×15 mL) and brine. The organic layer was dried over MgSO₄ and filtered. The filtrate was then concentrated under reduced pressure to give an off-white solid. The white solid was washed with n-hexane where starting material and the desired product was dissolved. The catalyst was filtered off. The remaining hexane solution was concentrated under reduced pressure to give a pale yellow liquid. Yield 58%; ¹H NMR (CDCl₃, δ) 7.13 (m, 2H), 6.73 (m, 1H), 4.26 (q, 2H), 3.93 (s, 1H), 1.74 (s, 3H), 1.29 (t, 3H).

The racemic compound 4 can be prepared in the absence of a chiral auxiliary using the similar procedure above. The R-enantiomer of compound 4 can be prepared in the presence of a (R) chiral auxiliary using the similar procedure above.

### Example 5

### Preparation of (S)-7-bromo-3-(3,5-difluorophenyl)-3-methyl-1H-pyrido[2,3-b][1,4]oxazin-2(3H)-one

### 5

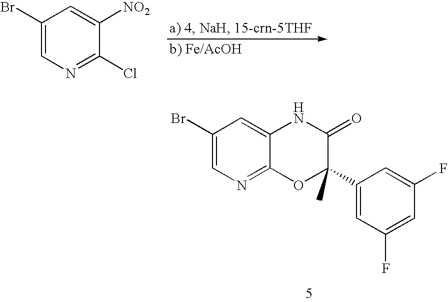

Step a) A solution of 2 g (8.7 mmol) of (S)-2-(3,5-difluorophenyl)-2-hydroxy-propionic acid ethyl ester (4 in anhydrous THF (25 mL) was treated with a 60% NaH dispersion in mineral oil by portions (0.35 g, 8.7 mmol). The grey suspension was resulted, which was stirred at room temperature for 10 min. Then 2-chloro-3-nitro-5-bromopyridine (1.65 g, 6.96 mmol) in 5 mL of dry THF was added. The resultant orange solution was stirred at room temperature for 6 h. Excess hydride was quenched by the careful addition of MeOH. The solution was diluted with EtOAc, and then washed with brine, dried over MgSO₄ which was filtered, and concentrated to dryness. A flash column chromatography was used to isolate the desired intermediate (hex 100% to 10% EtOAc/Hex). The intermediate was used for the next step without further purification. Step b) The residue was dissolved in 20 mL of glacial acetic acid, treated with 2.2 g (40 mmol) of iron dust, and heated in a 60° C. oil bath for 4 h. The resultant grey suspension was cooled to room temperature, diluted with EtOAc, and filtered through a Celite pad. The filtrate was concentrated to dryness. The solid residue was dissolved in EtOAc and washed with saturated aqueous NaHCO₃ and H₂O. The organic layer was dried over MgSO₄, which was filtered and concentrated to give an off-white solid. The pure desired product (5) was obtained via a recrystallization from MeOH and water. Yield 69%; ¹H NMR (CD₃OD, δ) 7.94 (s, 1H), 7.35 (s, 1H), 7.05 (d, 2H), 6.62 (m, 1H), 1.93 (s, 3H); MS (ESI+): m/z 354.0, 356.9 [M+1], Br isotopes, 1:1 ratio.

The racemic and R-enantiomer of compound 5 can be prepared using similar procedures.

### Example 6

### Preparation of 2-((S)-7-bromo-3-(3,5-difluorophenyl)-2,3-dihydro-3-methyl-2-oxopyrido[2,3-b][1,4]oxazin-1-yl)acetonitrile

### 6

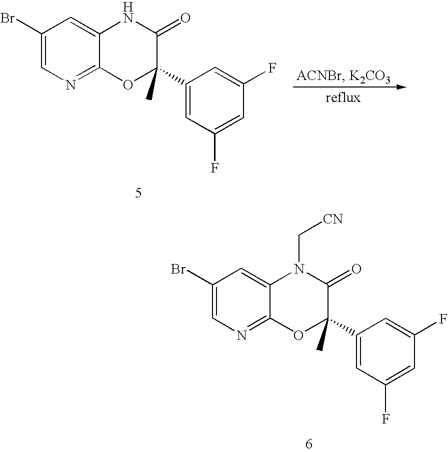

A solution of 5 (2.09 g, 5.89 mmol) in 30 mL of anhydrous acetonitrile was treated with 1.722 mL (11.8 mmol) of bromoacetonitrile and 2.0 g (14.78 mol) of K₂CO₃, and heated at reflux for 16 h. The resulting suspension was cooled to room temperature, diluted with EtOAc, and filtered through a Celite plug. The filtrate was washed with brine, dried over MgSO₄, and concentrated under reduced pressure. Purification by a flash column chromatography (SiO₂, 5% EtOAc/hexanes then gradient to 20% EtOAc/hexanes) gave 2.11 g as amorphous foam. Yield 91%; ¹H NMR (CDCl₃, δ ppm) 8.13 (s, 1H), 7.40 (s, 1H), 6.95 (d, 2H), 6.73 (m, 1H), 4.85 (dd, 2H), 1.93 (s, 3H); ¹³C NMR (CDCl₃, δ ppm) 164.9, 164.8, 161.6, 149.6, 143.9, 142.3, 124.7, 123.2, 114.3, 113.1, 108.5, 108.1, 104.9, 104.6, 104.3, 82.6; MS (ESI+): m/z 394.0, 396 [M+1], Br isotopes, 1:1 ratio.

The racemic and R-enantiomer of compound 6 can be prepared using similar procedures.

### Example 7

### Preparation of methyl 2-((S)-7-bromo-3-(3,5-difluorophenyl)-2,3-dihydro-3-methyl-2-oxopyrido[2,3-b][1,4]oxazin-1-yl)ethylcarbamate

### 7

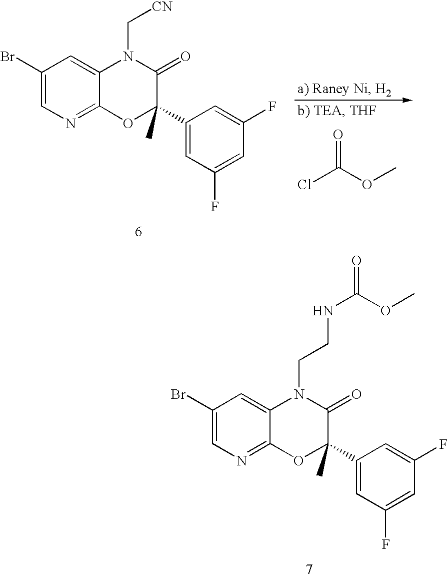

Methyl Cyanopyridoxazinone (6, 0.97 g) in 5 mL of dry THF was placed in a container (250 mL), which was charged with Raney Ni that was washed several times with dry THF (ca. 1 g in 10 mL THF). The reaction mixture was then supplied with H₂ gas in a balloon, and stirred at room temperature. Periodically, to the mixture was added methyl chloroformate and TEA. The progress of the reaction was monitored by TLC and addition of methyl chloroformate and TEA. The total reaction time was 24 h. The reaction mixture was filtered through a pad of Celite®, and the collected liquid was diluted with EtOAc (20 mL). The combined org. layer was concentrated under reduced pressure to give yellow amorphous foam. The desired product was isolated with a column chromatography (a gradient of 10% EtOAc to 25% in hex) 1.29 g. Yield 67%; ¹H NMR (CDCl₃, δ ppm) 8.13 (s, 1H), 7.60 (s, 1H), 6.95 (d, 2H), 6.73 (m, 1H), 4.15 (m, 1H), 3.90 (m, 1H), 3.65 (s, 3H), 3.40 (d, 2H), 1.93 (s, 3H); ¹³C NMR (CDCl₃, δ ppm) 165.5, 164.9, 161.6, 157.3, 143.4, 142.7, 125.2, 124.9, 123.2, 114.3, 108.5, 108.4, 108.1, 104.5, 104.2, 103.8, 82.6, 52.1, 41.4, 38.4, 37.4; MS (ESI+): m/z 455, 456 [M+1], Br isotopes, 1:1 ratio.

The racemic and R-enantiomer of compound 7 can be prepared using similar procedures.

### Example 8

### Preparation of Methyl 2-((S)-7-(2,4-diamino-6-ethylpyrimidin-5-yl)-3-(3,5-difluorophenyl)-2,3-dihydro-3-methyl-2-oxopyrido[2,3-b][1,4]oxazin-1-yl)ethylcarbamate

### 8

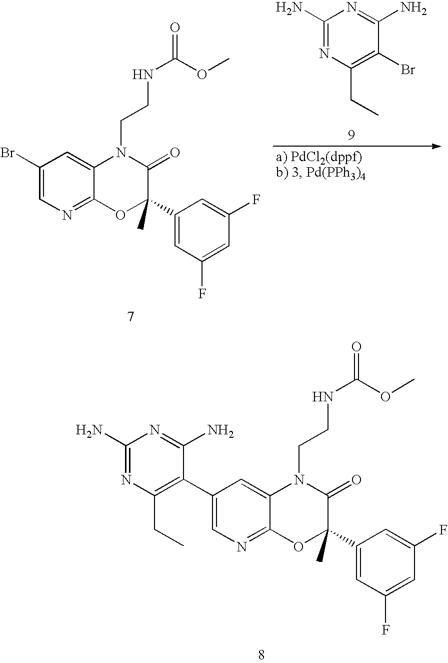

Step a) A solution of 7 (0.40 g, 0.877 mmol), bis(pinacolato)diboron (0.267 g, 1.05 mmol) and KOAc (0.258 g 2.63 mmol) in 10 mL of anhydrous 1,4-dioxane was degassed by applying a reduced pressure for 2 min while agitated. A PdCl₂(dppf)-CH₂Cl₂ complex (60 mg, 0.156 mmol) was added, and the resulting orange red suspension was heated in a 95° C. oil bath for 22 h. After cooling to room temperature, the black mixture was diluted with EtOAc, filtered through Celite, and concentrated.

Step b) The residue was dissolved in a mixture of 1,4-dioxane (10 mL) and H₂O (1.5 mL). To the solution, were added CsOH H₂O (0.445 g, 2.63 mmol 3 equiv), LiCl (0.119, 2.63 mmol, 3 equiv) and 5-bromo-6-ethylpyrimidine-2,4-diamine (9, 0.236 g, 1.10 mmol, 1.25 equiv). The resultant suspension was degassed by applying intermittent vacuum until no bubbling was observed. Pd(PPh₃)₄ (200 mg, 0.173 mmol) was then added to the reaction mixture, which was then heated to 100° C. for 18 h. The mixture was diluted with EtOAc (50 mL), dried over MgSO₄, filtered through a pad of Celite and the filtrate was concentrated to give a dark-brown oil. The desired product was obtained by a flash column chromatography using a gradient of 0 to 10% MeOH in DCM (120 mg). Yield 23%; ¹H NMR (CDCl₃, δ ppm) 7.93 (s, 1H), 7.65 (s, 1H), 7.30 (m, 1H), 7.25 (m, 1H), 6.99 (m, 1H), 4.25 (m, 2H), 3.60 (s, 3H), 3.55 (m, 2H), 2.37 2.20 (2 m, 2H rotamer), 2.05 (s, 3H), 1.17 1.00 (2 m, 3H rotamer); MS (ESI+): m/z 514 [M+1].

The racemic and R-enantiomer of compound 8 can be prepared using similar procedures.

### Example 9

### Preparation of Compounds 10-12

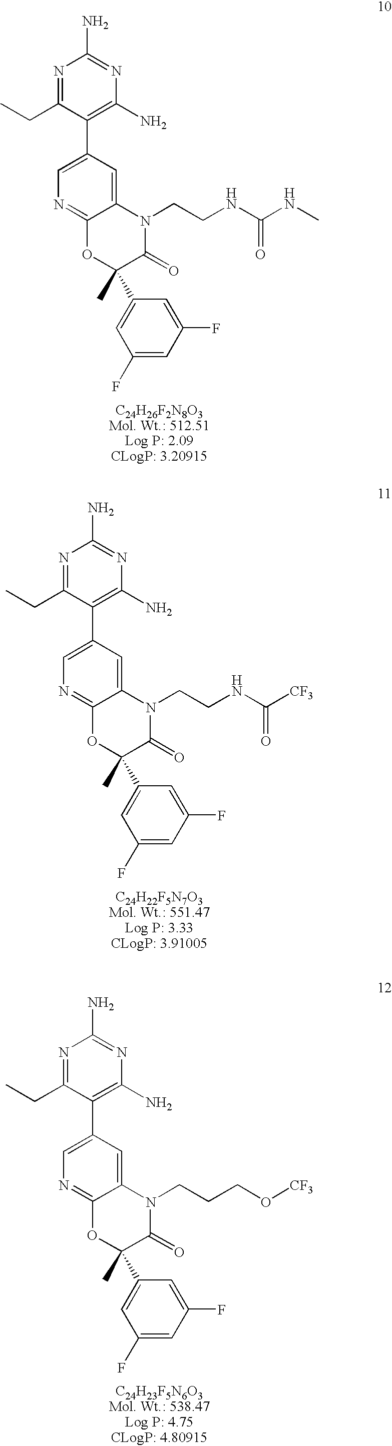

The compounds 10-12 were synthesized by the similar method as shown in Scheme 1. In particular, compounds 10 and 11 can be prepared from the same precursor 6 via the step a) reduction and the step b) with methyl isocyanate or trifluoroacetic anhydride as substrates to give the corresponding compound to 7 of 10 and 11, respectively.

### Example 10

### Preparation of Compound 13

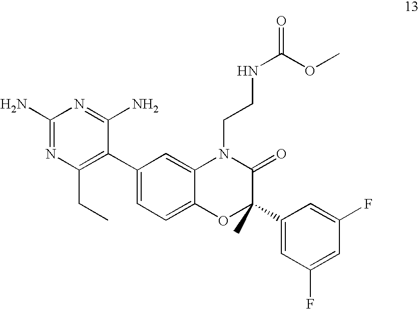

Compound 13 was synthesized by the similar method as shown in Scheme 1.

### Example 11

### In Vivo Model Studies

The following abbreviations are used:

IP: intraperitoneally
SHR: spontaneously hypertensive rat
BP: blood pressure

### Objective

The objective of this study is to define the magnitude, time course and dose/concentration response relationship of compound 13 antihypertensive effects when administered intraperitoneally (IP) to male SHR rats maintained on standard and low salt diets.

### Material and Methods

### Test Article

| | |
|---|---|
| Compound: | Compound 13 |
| Active Moiety (used for dose | 100% assumed |
| calculations): | |
| Vehicle: | 5% N-methylpyrrolidone (NMP)/45% |
| | PEG 400/50% 50 mM lactic acid |
| | (percentages based on volume) |
| Doses: | see table. |
| | Doses may be modified based upon |
| | results from previous dose(s) and from |
| | the dose selection phase of the study. |
| Dose Volume | 2.5 mL/Kg |
| Method of Administration: | intraperitoneal (see table) |

Dose Selection Rationale: Doses have been selected to assist in the evaluation of hemodynamic effects of compounds 13 and 8 in the absence of prior in vivo cardiovascular data in rat.

### Experimental Procedures

| | |
|---|---|
| Strain: SHR Rats | Sex: Male |
| Animal Numbers: 1-6 | Diet: Low sodium diet (through Treatment 8) |
| | then Standard rodent diet |

### Treatment Schedule—Phase I (Dose Selection Phase):

| Animal Number | Sex |
|---|---|
| 1 | M |
| 2 | M |
| 3 | M |

### Clinical Observations: Predose

### Instrumentation

| Instrument/Implant | Purpose |
|---|---|
| Radiotelemetry implant | Non-restrained cardiovascular data |
| | collection |

### Parameters to be Collected/Calculated:

Hemodynamic

| | |
|---|---|
| mean blood pressure systolic | mean blood pressure |
| blood pressure | systolic blood pressure |
| diastolic blood pressure heart rate | diastolic blood pressure heart rate |

Cardiovascular Data Collection: Through approximately 24 hours following each dose.

Disposition of Animals at Study Completion: Maintained for further studies when possible based upon acceptable functioning of telemetry device and health of animals.

### Description of Experimental Procedure

In Vivo Phase I: Three male SHR rats, 7-10 weeks of age weighing 150-300 g, will be instrumented with radiotelemetry transmitters. Following an approximate 2-week recovery period, animals will receive a single intraperitoneal dose of test article. Cardiovascular data will be collected for approximately 24 hours following dosing.

The methods to be used in this study are detailed in the departmental and In Vivo Center Standard Operating Procedures used at Michigan State University In Vivo Pharmacology Facility Study.

Telemetry cardiovascular Assessment in SHR Rats

Cardiovascular data is collected from non-restrained animals implanted with radiotelemetry devices.

### Equipment

Software: DSI Ponemah Physiology Platform (version 4.8), Dataquest OpenART Gold DSI telemetry implants, DSI receiver plates and other hardware.

### Procedures

1. SHR rats, 7-10 weeks of age weighing 150-300 g, are implanted with radiotelemetry devices. Transmitters are placed subcutaneously in the ventral abdominal region and blood pressure is monitored from the femoral artery. Animals will be allowed to recover at least 10 days prior to data collection.

2. Signal quality is verified by placing individual animals into a plastic solid-bottom cage set on top of a receiver plate.

3. Telemeters are turned on using a magnet. An AM radio is utilized to determine whether signals are being generated by the unit. Telemetry implants are turned off when data is not being collected in order to conserve battery power.

4. The software is set up according to the pertinent instruction manual.

5. At least 1 hour of baseline is collected from undisturbed animals prior to each dosing. Following test article administration, approximately 24 hours of data is collected from undisturbed animals to assess hemodynamic effects.

In a preliminary evaluation, blood pressure reduction in SHR is seen for compound 13.

1. A compound having formula (I): 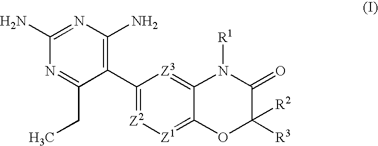
wherein
Z¹, Z² and Z³ are each independently selected from the group consisting of N, CH, C—CH₃ and C—OCH₃;
R¹ is a member selected from the group consisting of —(CH₂)ₙ—OR^{a}, (CH₂)ₙ—CF₃, —(CH₂)ₙ—CN, —(CH₂)ₙ—NHR^{b} and —(CH₂)ₙ—C(O)NHR^{a}, wherein R^{a} is a member selected from the group consisting of H, C₁₋₃ alkyl, C₁₋₃ hydroxyalkyl and C₁₋₃ haloalkyl; R^{b} is a member selected from the group consisting of —C(O)R^{c}, —C(O)OR^{c} and —S(O)₂R^{c}; and subscript n is an integer from 1-3, wherein R^{c} is selected from the group consisting of C₁₋₆ alkyl, C₁₋₆ hydroxyalkyl and C₁₋₆ haloalkyl; and wherein
(i) when at least one of Z¹, Z² and Z³ is N;
R¹ and R³ are each independently selected from the group consisting of C₁₋₆ alkyl, C₁₋₆ haloalkyl, substituted or unsubstituted pyridyl, substituted and unsubstituted phenyl, and substituted or unsubstituted piperidinyl;
(ii) when each of Z¹, Z² and Z³ is CH or C—CH₃;
R² is a member selected from the group consisting of methyl and trifluoromethyl; and R³ is a member selected from the group consisting of C₃₋₆ branched alkyl, C₃₋₆ branched haloalkyl, substituted or unsubstituted pyridyl, substituted and unsubstituted phenyl, and substituted or unsubstituted piperidinyl; wherein when R³ is substituted and unsubstituted phenyl, then R² is trifluoromethyl; or
(iii) when Z¹ is C—OCH₃;
R² and R³ are each independently selected from the group consisting of C₁₋₆ alkyl, C₁₋₆ haloalkyl, substituted or unsubstituted pyridyl, substituted and unsubstituted phenyl, and substituted or unsubstituted piperidinyl;
wherein the substituents on said pyridyl, phenyl and piperidinyl groups are selected from the group consisting of halo, C₁₋₆ alkoxy, C₁₋₆ alkyl, C₁₋₆ haloalkyl, C₁₋₆ alkylamino and di C₁₋₆ alkylamino;
and pharmaceutically acceptable salts and solvates thereof.

2. A compound of claim 1, wherein Z¹ is N.

3. A compound of claim 2, wherein R¹ is —(CH₂)ₙNHC(O)OR^{c} or —(CH₂)ₙNHC(O)NHR^{c}, —(CH₂)ₙNHC(O)R^{c} or —(CH₂)ₙNHR^{c}.

4. A compound of claim 3, wherein R^{c} is —CH₃ or —CF₃.

5. A compound of claim 3, wherein R¹ is —(CH₂)₂NHC(O)OR^{c} or —(CH₂)₂NHC(O)NHR^{c}, —(CH₂)₂NHC(O)R^{c} or —(CH₂)₃NHR^{c}.

6. A compound of claim 2, wherein R² is selected from the group consisting of methyl and trifluoromethyl; and R³ is selected from the group consisting of substituted or unsubstituted pyridyl and substituted or unsubstituted phenyl.

7. A compound of claim 6, wherein R³ is substituted or unsubstituted phenyl.

8. A compound of claim 7, wherein the substituents on the phenyl group is halo or C₁₋₆haloalkyl.

9. A compound of claim 8, wherein the substitutents on the phenyl group is fluoro.

10. A compound of claim 9, wherein R³ is 3,5-difluorophenyl.

11. A compound of claim 6, wherein R³ is selected from the group consisting of substituted or unsubstituted 3-pyridyl and substituted or unsubstituted 4-pyridyl.

12. A compound of claim 11, wherein R³ is selected from the group consisting of unsubstituted 3-pyridyl and unsubstituted 4-pyridyl.

13. A compound of claim 1, wherein Z¹ is CH.

14. A compound of claim 13, wherein Z² and Z³ are CH.

15. A compound of claim 13, wherein R² is selected from the group consisting of methyl and trifluoromethyl; and R³ is substituted or unsubstituted pyridyl.

16. A compound of claim 15, wherein R³ is selected from the group consisting of substituted or unsubstituted 3-pyridyl and substituted or unsubstituted 4-pyridyl.

17. A compound of claim 16, wherein R³ is selected from the group consisting of unsubstituted 3-pyridyl and unsubstituted 4-pyridyl.

18. A compound of claim 13, wherein R² is trifluoromethyl; and R³ is substituted or unsubstituted phenyl.

19. A compound of claim 18, wherein said substituted phenyl is selected from the group consisting of 3-methoxyphenyl and 4-methoxyphenyl.

20. A compound of claim 13, wherein R² is selected from the group consisting of methyl and trifluoromethyl; and R³ is selected from the group consisting of C₃₋₆ branched alkyl and C₃₋₆ branched haloalkyl.

21. A compound of claim 20, wherein R³ is isopropyl or tert-butyl.

22. A compound of claim 13, wherein R² is selected from the group consisting of methyl and trifluoromethyl; and R³ is substituted or unsubstituted piperidinyl.

23. A compound of claim 22, wherein R³ is substituted or unsubstituted 4-piperidinyl.

24. A compound of claim 23, wherein R³ is N-methylpiperidin-4-yl.

25. A compound of claim 1, wherein Z¹ is C—OCH₃

26. A compound of claim 25, wherein R¹ is selected from the group consisting of C₁₋₆ alkyl and C₁₋₆ haloalkyl; and R³ is selected from the group consisting of substituted or unsubstituted pyridyl, substituted and unsubstituted phenyl, and substituted or unsubstituted piperidinyl.

27. A compound of claim 26, wherein R² is selected from the group consisting of methyl and trifluoromethyl; and R³ is substituted or unsubstituted pyridyl.

28. A compound of any of claims 1, wherein the stereochemical configuration at the carbon atom bearing R² and R³ is (S).

29. A pharmaceutical composition comprising a pharmaceutically acceptable excipient and a compound of claim 1.

30. A method of treating renin-mediated diseases or conditions comprising administering to a subject in need of such treatment an effective amount of a compound of claim 1.

31. A method of claim 30, wherein the renin-mediated diseases or conditions are hypertension or congestive heart failure.
